(19) United States
(12) Patent Application Publication
(10) Pub. No.: US 2003/0153030 A1
(43) Pub. Date: Aug. 14, 2003

(54) Enzyme-catalyzed modification of substances in biological mixtures

(76) Inventors: Ralf Otto, Bad Friedrichshall (DE); Albrecht Weiss, Langenfeld (DE)

Correspondence Address: COGNIS CORPORATION, 2500 RENAISSANCE BLVD., SUITE 200, GULPH MILLS, PA, 19406

(21) Appl. No.: 10/168,579

(22) PCT Filed: Dec. 13, 2000
(86) PCT No.: PCT/EP00/12652

(30) Foreign Application Priority Data
Dec. 22, 1999 (DE) 199 62 204.3

(51) Int. Cl. C12P 1/00; C12N 9/00

(52) U.S. Cl. 435/41; 435/183

(57) ABSTRACT

The invention relates to an enzyme-catalysed modification of substances in a mixture, comprising bringing the substance in a mixture to be modified into contact with an enzyme and a substrate.

This invention relates to a process for the enzyme-catalyzed modification of substances in a mixture comprising contacting the substance to be modified in a mixture with an enzyme and a substrate.

In many biological, biotechnological and chemical processes, the products produced accumulate as mixtures of organic substances and can be extracted as such from the reaction mixture. By virtue of their better “dosability” and due to possible unwanted side effects of the other ingredients, individual substances of these extracts are often isolated and used, for example, in cosmetic preparations, cf. DE 19615577 A1, JP 11080002 A2. In addition, the isolated substances are frequently modified because it has been found that the activity of the substance can be increased by the modification. For example, arbutin, a skin-lightening glycoside from the bearberry, is up to 300 times more active in the form of its coumaroyl ester than unmodified arbutin, cf. EP 0 524 109 B1. Such improvements are also shown by salicin derivatives, such as p-OH-phenyl acetoyl salicin, as opposed to salicin. However, the desired substances are often difficult to isolate from the extracts. Since the ingredients generally have the polar character of the extractant in the extracts, elaborate chromatographic purification steps are necessary. For example, alcoholic extracts predominantly contain a mixture of hydrophilic components. In addition, the yield of the desired substance in such isolations is poor. Another disadvantage is that the other ingredients of the extracts are lost in the complicated working up processes.

On the other hand, however, the desired substances are able to develop their particular effect in synergism with the other ingredients of the extracts; cf. Lozoya, X., (1997) Spektrum der Wissenschaft, Sonderausgabe: Pharmaforschung, 6, 10-16. Because of this, plant extracts, for example, by virtue of the biologically active ingredients present in them are being increasingly used in the pharmaceutical and cosmetics industries without preliminary isolation of the desired substances; cf. Leung, A. Y., Foster, S., (1996, 2nd Edition), Encyclopedia of common natural ingredients—used in food, drugs and cosmetics, John Wiley and Sons, Inc., New York, Brisbane, Singapore. Here, too, there is a need to improve the activity of the desired substances by modification.

Accordingly, the problem addressed by the present invention was to provide a simple and efficient process for modifying substances in a mixture without preliminary isolation of the substance.

The solution to this problem as provided by the present invention is defined in the claims.

The expression “enzyme-catalyzed modification” as used herein means that a substance (target substance A) is coupled with a substrate (substance B) in a mixture by means of an enzyme as biocatalyst.

The invention is illustrated by the accompanying Figure.

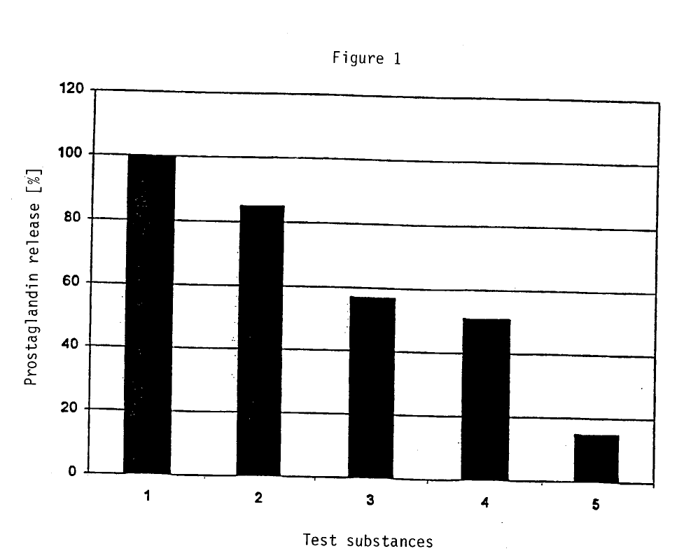

FIG. 1 is a schematic illustration of the effects of salicin and salicin esters on the release of prostaglandin in keratinocytes. The average value of three measuring points is shown. In FIG. 1, 1=negative control, 2=salicin, 3=phenyl propionyl salicin, 4=p-OH-phenyl acetyl salicin and 5=positive control.

One aspect of the present invention relates to a process for the enzyme-catalyzed modification of substances in a mixture comprising contacting the substance to be modified in a mixture with an enzyme and a substrate.

It has surprisingly been found that, by enzymatic, selective and nondestructive modification of the substances in mixtures, their biological activity can be increased without any effect on the other ingredients which remain intact as valuable substances. Biological activity and/or availability are improved and the solubility and/or affinity of the substance (target substance A) is/are changed. In addition, new substances with additional effects in relation to the original substance can be produced by the enzyme-catalyzed modification, for example by the coupling on of biologically active substrates (substance B). The target substance A can be hydrophobicized, for example by the coupling on of carboxylic acids with lipases, hydrophilicized by the coupling on of sugars/polyols with glycosidases or glycosyl transferases or modified in their affinity by the coupling on of substituted arylaliphatic carboxylic acids with lipases or by the coupling on of primary amines or peptides with transglutaminases or proteases or by the coupling on of polyols with glycosidases or glycosyl transferases.

The mixture may be an extract from vegetable, animal or microbial cells or may accumulate in biotechnological or chemical production processes. A process in which the vegetable, animal or microbial cell extract is liquid or dried is preferred. The modification of the substance may be carried out by adding to the mixture an enzyme which reacts the target substance A with a substrate B so that the desired modified substance A-B is formed. The enzyme may be present in free or immobilized form. Immobilized means that the enzyme (biocatalyst) may be coupled ionically or adsorptively to supports. The support may be a hydrophilic or hydrophobic particulate solid or a column matrix, the mixture being introduced via the column for the reaction. The support may also be a magnetized particle so that, after the reaction of the substance, the enzyme can be removed from the mixture by means of a magnetic field. In addition, the enzyme can be immobilized by crosslinking or incorporation in matrices or membranes. In addition, the enzyme may be polymer-derivatized.

The advantage over the chemical modification lies in the high selectivity, the mild conditions and the biocompatibility of the enzyme-catalyzed reaction. For example, glycoside modification by the coupling of carboxylic acids by standard chemical synthesis methods is known, cf. Colbert, J. C., Sugar Esters—Preparation and Application, Noyes Data Corporation, New Jersey (1974). The chemical preparation of esters from unprotected glycosides and carboxylic acids generally leads to nonspecific mixtures of mono- and polyacylated sugars so that protective groups have to be introduced and removed if a certain product is to be synthesized. However, the use of activated carboxylic acid derivatives, such as acid chlorides or anhydrides, leads to unwanted secondary products which pollute the environment, complicate working up and reduce the yields of the desired product.

A process in which the enzyme is selected from IUB enzyme classes 2 (transferases), 3 (hydrolases), 4 (lyases) or 6 (ligases) is preferred. A process in which the transferase is selected from the group of acyl, glycosyl, phosphoryl, methyl transferases or transglutaminases and the hydrolase is selected from the group of ester hydrolases, for example lipases or esterases, glycosidases, transglycosidases, epoxide hydrolases or proteases is particularly preferred. A cofactor-independent enzyme is particularly preferred. A cofactor-independent enzyme may be a lipase, for example, and may emanate from Candida parapsilopsis, Candida antarctica, Humicola lanuginosa, Rhizopus spec., Chromobacterium viscosum, Aspergillus niger, Candida rugosa, Geotrichum spec., Penicillium camembertii, Rhizomucor miehei, Burkholderia spec. or Pseudomonas spec. A cofactor-independent enzyme may also be an esterase or protease and may be of microbial or animal origin, emanating in particular from hog pancreas. A transglutaminase (Ca²⁺-dependent or independent), glycosidase or transglycosidase of microbial or animal origin is also particularly preferred. In addition, the enzyme used may be a synthetically produced or modified enzyme which contains one or more substitution(s), addition(s), deletion(s) or may be glycosylated.

The substrate may be any molecule which can be reacted by the enzyme used. A substrate with anti-inflammatory, antioxidative or antimicrobial properties is preferred. The substrate is preferably a carboxylic acid which may be activated or nonactivated, a primary amine or peptide corresponding to general formula (1) R′—NH₂, where R′ is an aliphatic or arylaliphatic radical containing at least 1 to 8 carbon atoms, or a sugar which may be activated or nonactivated.

Aromatic, aliphatic or arylaliphatic carboxylic acids which may consist of 2 to 26 carbon atoms and/or 1 to 10 hetero atoms and which may be substituted, unsubstituted, saturated or mono- or polyunsaturated are preferred. Particular preference is attributed to saturated aliphatic monocarboxylic acids, for example acetic acid, propionic acid, n-butyric acid, n-valeric acid, caproic acid, caprylic acid, capric acid, lauric acid, myristic acid, palmitic acid, stearic acid, hexacosanoic acid and mono- or polyunsaturated derivatives thereof, for example propenoic acid, crotonic acid, vinylacetic acid, palmitoleic acid, oleic acid, elaidic acid, linoleic acid, linolenic acid, eicosapentaenoic acid, stearidonic acid, arachidonic acid, conjugated linoleic acids and halo-, hydroxy- and nitro-substituted derivatives thereof. Aromatic and saturated or unsaturated arylaliphatic carboxylic acids, for example benzoic acid, phenylacetic acid, phenylpropionic acid, phenylbutyric acid, phenylvaleric acid and mono- or polyhydroxylated derivatives thereof, for example salicylic acid, m-, p-hydroxybenzoic acid, gallic acid, o-, m-, p-hydroxyphenylacetic acid, o-, m-, p-phenylpropionic acid, o-, m-, p-phenylbutyric acid, o-, m-, p-phenylvaleric acid, cinnamic acid, coumaric acids, coffee acid, are also particularly preferred.

Primary amines corresponding to general formula (1) R′—NH₂, where R′ is an aliphatic or arylaliphatic radical containing at least 1 to 8 carbon atoms or is substituted by hydroxy, halo or nitro groups, are also preferred. In addition, the amine may also be a peptide or polypeptide.

Other preferred substrates are activated or nonactivated sugars, for example threose, erythreose, arabinose, lyxose, ribose, xylose, allose, altrose, galactose, glucose, gulose, idose, mannose, talose or fructose and composite di- and oligomers and polymers thereof. The naturally occurring isomers of the sugars, particularly the D forms, are particularly preferred. N-acetyl glucosamine, sialic acid, vitamin C and uronic acids are also particularly preferred as are activated sugars containing an aglycone with good leaving group properties, for example halo, aryl (for example p-nitrophenyl), nucleoside, allyl, methyl or azide glycosides.

The process according to the invention may be carried out in the extracts themselves. The reaction temperature may be selected in any ranges in which the enzymes used are active. It is preferably carried out at temperatures in the range from 15 to 80° C., more preferably at temperatures in the range from room temperature to 80° C. and most preferably at temperatures in the range from room temperature to 60° C. Reaction temperatures in the range from room temperature to 45° C. are most particularly preferred.

The process according to the invention may be carried out in the absence of organic solvents. If it desired to add organic solvents, dioxane, acetonitrile, acetone, γ-butyrolactone, tetrahydrofuran, tert.butanol, tert.amyl alcohol or 3-methyl-3-pentanol, ethanol, methanol or carbonic acid esters, hexane or mixtures thereof, for example, may be used.

A preferred process is characterized in that, in the case of a hydrolysis reaction, the water formed during the esterification is removed from the system, for example with suitable molecular sieves or permeation membranes typically used or by application of a suitable reduced pressure.

In one preferred embodiment of the process according to the invention, the substance is removed from the mixture after the modification. It has surprisingly been found that, as a result of the nondestructive modification of substances using the process according to the invention, the properties of the substances can be altered so that the modified substance is far easier to isolate, i.e. is much easier to separate from the other components of the mixture than the unmodified substance. On completion of the reaction, the modified substance can be isolated from the reaction mixture by standard methods of isolation, for example by simple extraction with a suitable solvent or aqueous two-phase reaction using corresponding surfactants or polymers and/or chromatographic techniques using affinity and/or polarity. For example, the isolation of a glycoside from a hydrophilic plant extract can be greatly facilitated by coupling onto the glycoside by selective lipase catalysis a hydrophobic carboxylic acid which converts the glycoside into a lipophilic derivative.

By virtue of the modified polarity of the glycoside in relation to the other ingredients, the modified glycoside can readily be isolated from the extract by separation. This can be done by separation techniques known to the expert, for example by solvent extraction, a chromatographic process or recrystallization. Separation is preferably carried out by solvent extraction, the solvent used preferably being selected from nonpolar, substantially water-immiscible organic solvents, for example aliphatic or aromatic hydrocarbons containing 3 to 30 carbon atoms, ethers or esters containing 3 to 30 carbon atoms or halogenated hydrocarbons, for example methylene chloride or chloroform. Particularly preferred solvents are pentane, hexane, heptane, isooctane, methylene chloride, methyl tert.butyl ether and toluene. A substrate (anchor) with anti-inflammatory, antioxidative or antimicrobial properties is preferred. In a particularly preferred embodiment, the substrate is selected so that the substance is converted into an improved form in regard to its subsequent formulatability, stability and/or biological activity.

If desired, the substrate may readily be eliminated again after isolation of the modified substance by an enzymatic reversal reaction, for example a lipase- or protease- or glycosidase-catalyzed hydrolysis. A chemical reversal reaction may also be applied. A mild and selective enzymatic reaction is preferably used to eliminate the substrate.

### EXAMPLES

### Example 1

### Simple Isolation of Arbutin from a Commercially Obtainable Extract of Leaves of the Bearberry (Batch A) after Enzymatic Conversion into a Palmitoyl Ester

10 ml of a commercially obtainable ethanolic extract of leaves of the bearberry (Extractum Ursi Fluid, Chemische Fabrik Dr. Hetterich KG, Fürth; batch 04062098, contains at least 5.0% arbutin and other highly polar ingredients) were concentrated by evaporation and incubated with 500 mg palmitic acid, 2 g immobilized lipase (isoenzyme B from Candida antarctica), 5 g molecular sieve and 5 ml acetone at 45° C. in a rotating (75 r.p.m.) 50 ml round bottomed flask. The reaction of arbutin with palmitic acid was confirmed by thin-layer chromatography after 24 hours (silica gel 60 plates with fluorescence indicator; mobile solvent: chloroform/methanol/water 65:15:2 (v/v/v); visualization: UV detection and by acetic acid/sulfuric acid/anisaldehyde 100:2:1 (v/v/v) immersion reagent) by comparison with reference substance (R_{f} 0.39). The target product was separated from the other ingredients by simple extraction with methylene chloride or chloroform.

NMR of the isolated substance:

¹³C-NMR (CD₃OD): δ(ppm)=14.4 (C-16), 23.7 (C-15), 26.0 (C-3), 30.0-33.0 (C-4-C-14), 35.1 (C-2), 64.7 (C-6′), 71.7 (C-4′), 74.8 (C-2′), 75.3 (C-5′), 77.8 (C-3′), 103.6 (C-1′), 116.6 (C-3*, C-5*), 119.5 (C-2*, C-6*), 152-154 (C-1*, C-4*), 175.2 (C═O). (Marking: none=acyl group, ′=glucose, *=aglycone)

### Example 2

### Simple Isolation of Arbutin from a Commercial Extract of Leaves of the Bearberry (Batch B) after Enzymatic Conversion into a Phenylpropionyl Ester

5 ml of a commercially obtainable extract of leaves of the bearberry (Extractum Ursi Fluid, Chemische Fabrik Dr. Hetterich KG, Fürth; batch 01810797, contains at least 5.7% arbutin) were concentrated by evaporation and incubated with 2 g immobilized lipase (isoenzyme B from Candida antarctica), 5 g molecular sieve and 5 ml t-butanol as solvent at 60° C. in a rotating (75 r.p.m.) 50 ml round bottomed flask. Phenylpropionic acid was added in steps (50 mg every 4 hours) to avoid excesses of acid in the mixture. The reaction of arbutin with phenylpropionic acid was confirmed by thin-layer chromatography after 24 hours (silica gel 60 plates with fluorescence indicator; mobile solvent: chloroform/methanol/water 65:15:2 (v/v/v); visualization: UV (254 nm) detection and by acetic acid/sulfuric acid/anisaldehyde 100:2:1 (v/v/v) immersion reagent) by comparison with reference substance (6-O-phenylpropionyl-[4-(hydroxyphenyl))]-□-D-glucopyranoside, R_{f} 0.33). The target product was isolated by simple extraction with methylene chloride or even chloroform.

NMR of the isolated substance:

¹³C-NMR (CD₃OD): δ(ppm)=31.9 (C-2), 36.9 (C-3), 64.8 (C-6′), 71.7 (C-4′), 74.9 (C-2′), 75.3 (C-5′), 77.8 (C-3′), 103.5 (C-1′), 116.7 (C-3*, C-5*), 119.7 (C-2*, C-6*), 127.2 (C-7), 129.4 (C-5, C-6, C-8, C-9), 141.8 (C-4), 152.2, 153.9 (C-1*, C-4*), 174.2 (C═O). (Marking: none=acyl group, ′=glucose, *=aglycone)

### Example 3

### Elimination of the Substrate (Anchor)

5 mg of the isolated palmitoyl arbutin (Example 1) was dissolved in phosphate buffer (pH 7.4, 0.1 M) and incubated for 2 h with 5 mg Candida antarctica Lipase B (SP 435). The course of the elimination process was followed by thin-layer chromatography (silica gel 60 plates with fluorescence indicator; mobile solvent: chloroform/methanol/water 65:15:2 (v/v/v); visualization: UV detection and by acetic acid/sulfuric acid/anisaldehyde 100:2:1 (v/v/v) immersion reagent). After 1 hour, the ester was completely hydrolyzed.

### Example 4

### Enzymatic Modification of Salicin in Willow Bark Extracts

10 ml of a commercially obtainable willow bark extract (Extractum Salicis, Chemische Fabrik Dr. Hetterich KG, Fürth; contains at least 2.5% salicin) were concentrated by evaporation and incubated with 2 g immobilized lipase (isoenzyme B from Candida antarctica), 5 g molecular sieve, 500 mg phenylpropionic acid and 5 ml t-butanol as solvent at 60° C. in a rotating (75 r.p.m.) 50 ml round bottomed flask. The reaction of salicin with phenylpropionic acid was confirmed by thin-layer chromatography after 24 hours (silica gel 60 plates with fluorescence indicator; mobile solvent: chloroform/methanol/water 65:15:2 (v/v/v); visualization: UV (254 nm) detection and by acetic acid/sulfuric acid/anisaldehyde 100:2:1 (v/v/v) immersion reagent). The target product was isolated by simple extraction with methylene chloride or even chloroform.

NMR of the isolated substance:

¹³C-NMR (CD₃OD): δ(ppm)=30.9 (C-2), 38.7 (C-3), 61.6 (C-7*), 65.2 (C-6′), 72.2 (C-4′), 75.6 (C-2′), 76.1 (C-5′), 78.4 (C-3′), 103.7 (C-1′), 117.6 (C-6*), 124.5 (C-4*), 127.6 (C-7), 130.1-131.0 (C-3*, C-5*, C-5, C-6, C-8, C-9), 132.8 (C-2*), 143.4 (C-4), 157.5 (C-1*), 175.2 (C═O). (Marking: none=acyl group, ′=glucose, *=aglycone)

### Example 5

### Introduction of Additional Effective Partial Structures into Ingredients of Willow Bark

Arylaliphatic carboxylic acids are known for their antioxidative and antibacterial effects. In addition, p-hydroxylated phenylacetic acid has anti-inflammatory activity. 10 ml of a commercially obtainable willow bark extract (Extractum Salicis, Chemische Fabrik Dr. Hetterich KG, Fürth; contains at least 2.5% salicin) were concentrated by evaporation and incubated with 2 g immobilized lipase (isoenzyme B from Candida antarctica), 5 g molecular sieve, 500 mg p-OH-phenylacetic acid and 5 ml t-butanol as solvent at 60° C. in a rotating (75 r.p.m.) 50 ml round bottomed flask. The reaction of salicin with p-OH-phenylacetic acid was confirmed by thin-layer chromatography after 24 hours (silica gel 60 plates with fluorescence indicator; mobile solvent: chloroform/methanol/water 65:15:2 (v/v/v); visualization: UV (254 nm) detection and by acetic acid/sulfuric acid/anisaldehyde 100:2:1 (v/v/v) immersion reagent). For NMR analysis, the target product was isolated by simple extraction with methylene chloride or even chloroform.

NMR of the isolated substance:

¹³C-NMR (CD₃OD): δ(ppm)=41.8 (C-2), 61.0 (C-7*), 65.0 (C-6′), 71.5 (C-4′),74.9 (C-2′), 75.4 (C-5′), 77.8 (C-3′), 103.2 (C-1′), 117.1 (C-6*), 123.9 (C-4*), 129.4-132.3 (C-2*, C-3*, C-5*, C-4, C-5, C-7, C-8), 136.1 (C-3), 156.0-159.2 (C-1*, C-6), 173.31 (C═O). (Marking: none=acyl group, ′=glucose, *=aglycone)

### Example 6

Murine (MSCP5) and human (HPKII) skin keratinocytes were marked for 16 hours with 0.2 μCi ¹⁴C-arachidonic acid/ml medium. Salicin, phenylpropionyl salicin and p-OH-phenylacetyl salicin in fresh medium were added in increasing concentrations as test substances and incubated for 2 hours. In the positive control NS398 (10 μM) for MSCP5 cells, prostaglandin synthesis is reduced by 85%. The prostaglandins were identified by comparison with reference substances and quantified by radiodensitometry. MSCP5: 100%=201 cpm; HPKII: 100%=63 cpm.

1. A process for the enzyme-catalyzed modification of substances in a mixture comprising contacting the substance to be modified in a mixture with an enzyme and a substrate.

2. A process as claimed in claim 1, characterized in that the mixture is a liquid or dried vegetable, animal or microbial cell extract.

3. A process as claimed in claim 1 or 2, characterized in that the enzyme is selected from IUB enzyme classes 2 (transferases), 3 (hydrolases), 4 (lyases) or 6 (ligases)

4. A process as claimed in claim 3, characterized in that the transferase is selected from the group of acyl, glycosyl, phosphoryl, methyl transferases or transglutaminases and the hydrolase is selected from the group of ester hydrolases (lipases or esterases), glycosidases, transglycosidases, epoxide hydrolases or proteases.

5. A process as claimed in any of claims 1 to 4, characterized in that the enzyme is a cofactor-independent enzyme.

6. A process as claimed in claim 5, characterized in that the cofactor-independent enzyme is a lipase and emanates from Candida parapsilopsis, Candida antarctica, Humicola lanuginosa, Rhizopus spec., Chromobacterium viscosum, Aspergillus niger, Candida rugosa, Geotrichum spec., Penicillium camembertii, Rhizomucor miehei, Burkholderia spec. or Pseudomonas spec. or is an esterase or protease and is of microbial or animal origin, emanating in particular from hog pancreas.

7. A process as claimed in claim 4, characterized in that the transglutaminase, glycosidase and transglycosidase is of microbial or animal origin and the transglutaminase is Ca²⁺-dependent or -independent.

8. A process as claimed in any of claims 1 to 7 characterized in that the enzyme is immobilized on a support.

9. A process as claimed in any of claims 1 to 8, characterized in that the substrate is an activated or nonactivated carboxylic acid, a primary amine or peptide corresponding to general formula (1) R′—NH₂, where R′ is an aliphatic or arylaliphatic radical containing at least 1 to 8 carbon atoms, or an activated or nonactivated sugar.

10. A process as claimed in any of claims 1 to 9, characterized in that it is carried out at temperatures in the range from 15 to 80° C., preferably at temperatures in the range from room temperature to 80° C., more preferably at temperatures in the range from room temperature to 60° C. and most particularly at temperatures in the range from room temperature to 45° C.

11. A process as claimed in any of claims 1 to 10, characterized in that it further comprises isolating the modified substance from the mixture.

12. A process as claimed in claim 11, characterized in that the modified substance is isolated by extraction with an organic solvent.

13. A process as claimed in claim 11 or 12, characterized in that it further comprises eliminating the substrate from the modified substance.

14. A cosmetic, pharmaceutical or food preparation comprising at least one substance obtainable by the process claimed in any of claims 1 to 13.
